(19) United States
(12) Patent Application Publication
(10) Pub. No.: US 2017/0343634 A1
(43) Pub. Date: Nov. 30, 2017

(54) USE OF STRIATAL CONNECTIVITY PATTERNS FOR EVALUATING ANTIPSYCHOTIC AGENTS

(71) Applicant: THE FEINSTEIN INSTITUTE FOR MEDICAL RESEARCH, Great Neck, NY (US)

(72) Inventors: Todd Lencz, New York, NY (US); Anil K. Malhotra, Rye Brook, NY (US); Deepak K. Sarpal, Astoria, NY (US); Miklos Argyelan, Bayville, NY (US); Delbert Robinson, New York, NY (US)

(21) Appl. No.: 15/610,690

(22) PCT Filed: Nov. 30, 2015
(86) PCT No.: PCT/US15/62927

§ 371 Date: Jun. 1, 2017

Related U.S. Application Data
(60) Provisional application No. 62/085,707, filed on Dec. 1, 2014.

(51) Int. Cl. G01R 33/48; A61B 5/055; A61B 5/00

(52) U.S. Cl. CPC G01R 33/4806; A61B 5/7264; A61B 5/0042; A61B 5/055; A61B 5/4848

(57) ABSTRACT

A method of predicting the response of a subject to an antipsychotic agent is described. The method includes obtaining functional MRI (fMRI) scan data of the brain of the subject, modifying the scan data using a standardizing algorithm to provide modified scan data, calculating the value of a plurality of striatal connectivity dyads from the modified scan data using an extraction algorithm, calculating a combined score from the values of the striatal connectivity dyads using a combining algorithm; and comparing the combined score to a classifier value to determine if the subject is a responder or a non-responder. Systems for carrying out the method of predicting the response of a subject to an antipsychotic agent are also described.

### CROSS-REFERENCE TO RELATED APPLICATIONS

This application claims priority to U.S. Provisional Application Ser. No. 62/085,707, filed on Dec. 1, 2014, which is hereby incorporated by reference in its entirety.

### STATEMENT REGARDING FEDERALLY SPONSORED RESEARCH

This invention was made, at least in part, with government support under Grant No. P50MH080173, Grant No. P30MH090590, Grant No. R01MH060004, and Grant No. R01MH076995 awarded by the National Institutes of Health. The government has certain rights in the invention.

### BACKGROUND

Chronic psychotic disorders are estimated to occur in over 3% of the population and contribute a considerable amount of morbidity worldwide. According to The Global Burden of Diseases, Injuries, and Risk Factors Study of 2010, two of these illnesses, schizophrenia and bipolar disorder, accounted for over 27 million years lived with disability. Schizophrenia has been shown to reduce the lifespan of sufferers by 12-15 years due to factors such as substance use, poverty, neglect of personal well-being, and suicide.

Antipsychotic medications are the mainstay for treatment of psychosis, yet they are associated with substantial heterogeneity in their therapeutic efficacy. Conley R R, Kelly D L., Biol Psychiatry, 50: 898-911 (2001); Leucht et al., The Lancet, 382: 951-62 (2013). Non-response to standard medications contributes to poor functional outcomes and a large economic impact on healthcare systems, including up to a 10-fold increase in total health resource utilization. Treatment algorithms for these illnesses are devoid of prognostic measures, and clinicians often resort to trial-and-error when faced with the potential inefficacy of their medication choices. Moreover, when patients fail to show an adequate clinical response to standard agents, they endure prolonged periods of untreated illness. There is some evidence that non-response within the first 2 weeks of treatment may ultimately predict a failed medication trial, yet even this approach requires costly trial-and-error. Leucht et al., J Clin Psychiatry 68: 352-60 (2007); Correll et al., Am J Psychiatry, 160: 2063-5 (2003). Moreover, this finding is limited to chronic sufferers; medication trials in patients with first-episode schizophrenia (FES) are recommended to last up to 16 weeks. Increased time of unremitted illness during medication trials alone results in doubled health care costs relative to remitted illness, and an increased strain on patients and their families, resulting in a diminished alliance with overall psychiatric care.

Current practice suggests a need for reliable, biologically based, prognostic measures of treatment response to antipsychotic agents. In the domain of neuroimaging, structural methods have shown that alterations in cortical thickness, asymmetry, and gyrification may be associated with subsequent response to treatment with antipsychotics. Szeszko et al. Schizophr Bull 38: 569-78 (2012); Palaniyappan et al., JAMA Psychiatry 70: 1031-40 (2013). Reduced white matter integrity has also been linked to non-response to treatment with antipsychotic agents in patients with first episode psychosis. Marques et al., Brain, 137: 172-82 (2014). Though important in explicating pathophysiologic mechanisms, these findings have not been replicated in independent cohorts, and have not resulted in predictive tests with clinical utility.

Multiple lines of evidence suggest that variation in physiology of the striatum may be critical to antipsychotic treatment outcomes. This subcortical region contains a dense concentration of dopamine D2 receptors, the shared target of all known antipsychotic agents. While genetic variation at the dopamine D2 receptor has replicably been shown to influence response to these medications, the modest size of this effect limits clinical utility. Zhang et al., Am J Psychiatry 167: 763-72 (2010). Treatment-resistant schizophrenia has been associated with normal dopaminergic synthesis capacity in the striatum, while treatment responders demonstrate elevated striatal dopamine in psychotic illness. Howes et al., Br J Psychiatry 205: 1-3 (2014); Demjaha et al., Am J Psychiatry 169: 1203-10 (2012). In congruence with cross-sectional data that has linked abnormal corticostriatal interactions with psychotic illness (Meyer-Lindenberg et al., Nat Neurosci 5: 267-71 (2002); Fornito et al. JAMA Psychiatry; 70: 1143-51 (2013), we recently reported that improvement of psychotic symptoms is associated with changes in striatal functional connectivity over the course of treatment. Sarpal et al., JAMA Psychiatry 72(1):5-13 (2015). We hypothesized that alterations in functional connectivity of the striatum may provide prognostic value in the treatment of psychosis.

### SUMMARY OF THE INVENTION

In one aspect, the present invention provides a method of predicting the response of a subject to an antipsychotic agent. The method includes the steps of obtaining functional MRI (fMRI) scan data of the brain of the subject; modifying the scan data using a standardizing algorithm to provide modified scan data; calculating the value of a plurality of striatal connectivity dyads from the modified scan data using an extraction algorithm; calculating a combined score from the values of the striatal connectivity dyads using a combining algorithm; and comparing the combined score to a classifier value to determine if the subject is a responder or a non-responder. In some embodiments, the method also includes the step of obtaining the fMRI scan data by conducting an fMRI scan of the subject using an fMRI imaging apparatus. In further embodiments, the seed voxel of one or more of the striatal connectivity dyads is found in a brain region selected from the group consisting of the insula cortex, the opercular cortex, the anterior cingulate, the thalamus, the orbitofrontal cortex, and the precuneus regions.

In some embodiments, the subject has been diagnosed as having a psychotic disorder selected from the group consisting of schizophrenia, schizophreniform disorder, schizoaffective disorder, delusional disorder, shared psychotic disorder, brief psychotic disorder, psychotic disorder due to a general medical condition, substance-induced psychotic disorder, bipolar I disorder (with psychotic features) and major depressive disorder (with psychotic features). In other embodiments, the subject has been diagnosed as having a non-psychotic disorder selected from the group consisting of bipolar I disorder (acute treatment of manic, mixed, or depressive episodes; maintenance treatment), major depressive disorder, Irritability associated with autistic disorders, agitation associated with schizophrenia or bipolar mania, and irritability associated with autistic disorders.

Determining responder or non-responder status can be used to guide subsequent treatment of the subject. In some embodiments, the method includes administering a therapeutically effective amount of a non-clozapine atypical antipsychotic to the subject if the subject is identified as a responder. In other embodiments, the method includes administering a therapeutically effective amount of clozapine to the subject if the subject is identified as a non-responder.

Another aspect of the invention provides a system for predicting a response of a patient to a given antipsychotic drug. The system includes a medical diagnostic scanner configured to provide a spatial representation of neural activity within the brain; a feature extractor configured to extract a set of striatal connectivity dyads representing the functional connectivity of specified nodes in the basal ganglia to other specified areas of the brain; a classifier configured to classify the patient into one of a plurality of classes representing the likelihood that the patient will respond to the antipsychotic drug from the extracted set of striatal connectivity dyads; and an output device configured to provide the resulting classification to a user in human comprehensible form.

In some embodiments, the system includes a preprocessing component configured to condition the spatial representation of neural activity within the brain for analysis. In additional embodiments, the medical diagnostic scanner is configured to perform a functional magnetic resonance imaging scan. In yet further embodiments, the classifier performs a binary classification, such that the patient is classified into either a “responder” or a “non-responder” class.

### BRIEF DESCRIPTION OF THE FIGURES

The present invention may be more readily understood by reference to the following figures, wherein:

FIG. 1 provides a scheme showing steps involved in the method of predicting the response of a subject to an antipsychotic agent.

FIG. 2 provides a schematic representation of a system 400 for predicting a patient response to a given antipsychotic drug.

FIG. 3 provides a schematic representation of a computer system 500 that can be employed to implement systems and methods described herein, such as based on computer executable instructions running on the computer system.

FIG. 4 provides an image in which the functional connections with our striatal regions of interest that showed predictive value and were included in the computation of our prognostic score are illustrated. Connections that positively predict response to treatment are in dark gray, while connections that negatively predict response are in light gray.

FIGS. 5A-5C provide graphs in which the results of our prognostic test are displayed in our discovery and replication cohorts (5A). Receiver operating curves that correspond with our results are displayed for our discovery cohort in 5B, and for our replication cohort in 5C.

FIG. 6 provides a graph in which the length of stay is plotted against our striatal functional connectivity score. Size of dot indicates baseline BRPS-A total score.

### DETAILED DESCRIPTION OF THE INVENTION

The present invention relates to methods of predicting the response of a subject to an antipsychotic agent. The method includes obtaining functional MRI (fMRI) scan data of the brain of the subject, modifying the scan data using a standardizing algorithm to provide modified scan data, calculating the value of a plurality of striatal connectivity dyads from the modified scan data using an extraction algorithm, calculating a combined score from the values of the striatal connectivity dyads using a combining algorithm; and comparing the combined score to a classifier value to determine if the subject is a responder or a non-responder. The invention also relates to systems for carrying out the method of predicting the response of a subject to an antipsychotic agent.

### Definitions

As used herein, the term “diagnosis” can encompass determining the likelihood that a subject will develop a disease, or the existence or nature of disease in a subject.

As used herein, the term “prognosis” refers to a prediction of the likelihood that treatment will be successful, or the likelihood of recovery from a disease subsequent to treatment. Prognosis is distinguished from diagnosis in that it is generally already known that the subject has a disease, although prognosis and diagnosis can be carried out simultaneously. In the case of a prognosis for treatment of psychosis, the prognosis categorizes the nature of the psychosis, which can be used to guide selection of appropriate therapy using antipsychotic agents.

As used herein, the terms “treatment”, “treating”, and the like, refer to obtaining a desired pharmacologic or physiologic effect. The effect may be therapeutic in terms of a partial or complete cure for a disease or an adverse effect attributable to the disease. “Treatment”, as used herein, covers any treatment of a disease in a mammal, particularly in a human, and can include inhibiting the disease or condition, i.e., arresting its development; and relieving the disease, i.e., causing regression of the disease.

The terms “therapeutically effective” and “pharmacologically effective” are intended to qualify the amount of an agent which will achieve the goal of improvement in disease severity and the frequency of incidence. The effectiveness of treatment may be measured by evaluating a reduction in psychotic symptoms in a subject in response to the administration of antipsychotic agents.

The terms “individual”, “subject”, and “patient” are used interchangeably herein irrespective of whether the subject has or is currently undergoing any form of treatment. As used herein, the term “subject” generally refers to a mammal that is capable of developing psychosis. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Predicting the effect of psychotic agents in humans is of particular interest.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” refers to +/−10% deviation from the basic value.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “a connectivity dyad” includes reference to one or more connectivity dyads, and so forth.

### Methods of Predicting the Response of a Subject to an Antipsychotic Agent

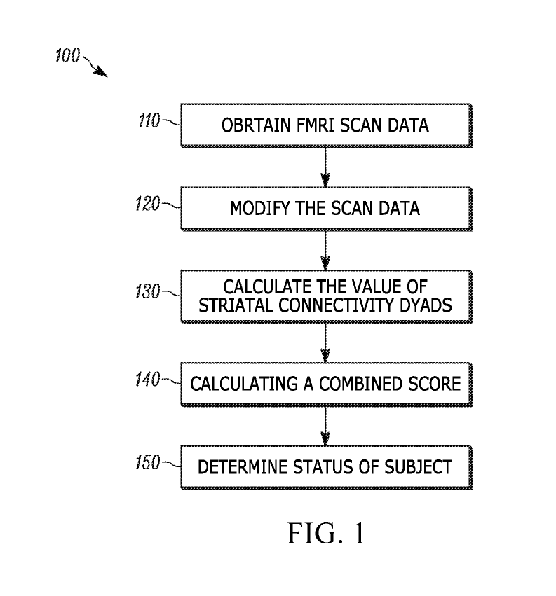

One aspect of the invention provides a method of predicting the response of a subject to an antipsychotic agent. The steps involved in the method 100 of predicting the response of a subject to an antipsychotic agent are shown in FIG. 1. The method includes the steps of obtaining functional MRI (fMRI) scan data of the brain of the subject 110; modifying the scan data using a standardizing algorithm to provide modified scan data 120; calculating the value of a plurality of striatal connectivity dyads from the modified scan data using an extraction algorithm 130; calculating a combined score from the values of the striatal connectivity dyads using a combining algorithm 140; and comparing the combined score to a classifier value to determine if the subject is a responder or a non-responder 150.

A wide variety of antipsychotic agents are known to those skilled in the art. Antipsychotics, which are also known as neuroleptics or major tranquilizers, are a class of psychiatric medication primarily used to manage psychosis, which includes delusions, hallucinations, or disordered thought, and in particular in schizophrenia and bipolar disorder. Antipsychotic agents can be roughly divided into first generation antipsychotics (i.e., typical antipsychotics) and second generation antipsychotics (i.e. atypical antipsychotics). Examples of typical antipsychotics include chlorpromazine, chlorprothixene, levomepromazine, mesoridazine, periciazine, promazine, thioridazine, loxapine, molindone, perphenazine, thiothixene, droperidol, flupentixol, fluphenazine, haloperidol, pimozide, prochlorperazine, thioproperazine, trifluoperazine, and zuclopenthixol. Examples of atypical antipsychotics include amisulpride, aripiprazole, asenapine, blonanserin, clozapine, iloperidone, lurasidone, melperone, olanzapine, paliperidone, quetiapine, risperidone, sertindole, sulpride, ziprasidone, and zotepine.

Various steps in the method 100 of predicting the response of a subject to an antipsychotic agent are shown in FIG. 1. Process block 110 represents the step of obtaining fMRI scan data. Initially, time-series fMRI data is provided. In some aspects, this step may also include acquiring a set of time-series fMRI data using a magnetic resonance imaging (MRI) system. In some embodiments, this fMRI data may be indicative of a resting state of the subject. The fMRI scan data can have been previously obtained, or the method can include the step of obtaining the fMRI scan data by conducting an fMRI scan of the subject using an fMRI imaging apparatus.

Functional MRI is a variation of Magnetic Resonance Imaging (MRI). MRI brain scans use a strong, permanent, static magnetic field to align nuclei in the brain region being studied. Another magnetic field, the gradient field, is then applied to spatially locate different nuclei. Finally, a radiofrequency (RF) pulse is played to kick the nuclei to higher magnetization levels, with the effect now depending on where they are located. When the RF field is removed, the nuclei go back to their original states, and the energy they emit is measured with a coil to recreate the positions of the nuclei. MRI thus provides a static structural view of brain matter.

fMRI allows MRI to capture functional changes in the brain caused by neuronal activity based on differences in magnetic properties between arterial (oxygen-rich) and venous (oxygen-poor) blood. See U.S. patent application Ser. No. 14/672,657. When neurons go active, getting them back to their original (polarized) state requires actively pumping ions back and forth across the neuronal cell membranes. The energy for those ion pumps is mainly produced from glucose. More blood flows in to transport more glucose, also bringing in more oxygen in the form of oxygenated hemoglobin molecules in red blood cells. The blood-flow change is localized to within 2 or 3 mm of where the neural activity is. The newly introduced oxygen is more than the oxygen consumed in burning glucose, causing a net decrease in deoxygenated hemoglobin (dHb) in that brain area's blood vessels. Hemoglobin differs in how it responds to magnetic fields, depending on whether it has a bound oxygen molecule. The dHb molecule is more attracted to magnetic fields. Hence, it distorts the surrounding magnetic field induced by an MRI scanner, causing the nuclei there to lose magnetization faster via the T2* decay. Thus MR pulse sequences sensitive to T2* show more MR signal where blood is highly oxygenated and less where it is not. This form of fMRI is called the blood-oxygen-level dependent (BOLD) contrast. Huettel et al., “Functional Magnetic Resonance Imaging Second Edition”, 2009, Massachusetts: Sinauer, ISBN 978-0-87893-286-3.This effect increases with the square of the strength of the magnetic field. The fMRI signal hence needs both a strong magnetic field (1.5 T or higher) and a pulse sequence such as EPI, which is sensitive to T2* contrast. Images of the whole brain, or sections (slices) of brain, are obtained very rapidly (in seconds) and repeatedly over a period of several minutes, resulting in a time series of brain images.

Functional imaging procedures can be used to map brain activity in quiet, resting-state subjects, and to explore the connectivity of brain systems. Such methods take advantage of spontaneous brain activity events that cascade through all brain systems and thus provide insight into the normally functioning brain as well as the abnormalities due to brain diseases. Functional brain organization of each individual subject can be determined based on resting-state using an iterative adjusting approach. The iterative optimization process can be guided by a population-based functional atlas.

The apparatus used to carry out fMRI includes a magnet to generate a static magnetic field B₀, gradient coils and power supplies to generate linear magnetic field gradients along the X, Y and Z axes, shim coils and shim power supplies to generate higher order magnetic field gradients, single or multiple radiofrequency (RF) transmit coils and RF transmitter to generate an RF field, single or multiple RF receiver coils forming an array, RF receivers and digitizers to measure the received RF field, and a computer to generate the pulse sequence and to control the components of the MRI apparatus, as well a computer to measure and reconstruct the MR signals, and a computer to analyze the reconstructed images. The three computers mentioned immediately above may be the same computer or may be realized in different computers. The methods described in this application refer to processes involved in analyzing reconstructed images (scan data).

The scanner platform generates a 3D volume of the subject's head every time resolution (TR), which is how often a particular brain slice is excited and allowed to lose its magnetization. This consists of an array of voxel intensity values, one value per voxel in the scan. The voxels are arranged one after the other, unfolding the three-dimensional structure into a single line. Several such volumes from a session are joined together to form a 4D set of volumes corresponding to a run, for the duration of that particular scanning sequence. This 4D set of volumes is the starting point for analysis.

At process block 120, the scan data obtained in step 110 is modified using a standardizing algorithm to provide modified scan data. Examples of standardized algorithms for modifying scan data are, for example, the FMRIB Software Library (FSL) at the Oxford Centre for Functional MRI of the Brain and Analysis of Functional Neurolmages (AFNI) scripts at the National Institute of Mental Health. These preprocessing steps are standard in the art of fMRI analysis.

One step in modifying the scan data is conventionally slice timing correction. The magnetic resonance scanner acquires different slices within a single brain volume at different times, and hence the slices represent brain activity at slightly different timepoints. Since this complicates later analysis, a timing correction is applied to bring all slices within a single acquired volume to the same timepoint reference. This is done by assuming the timecourse of a voxel is smooth when plotted as a dotted line. Hence the voxel's intensity value at other times not in the sampled frames can be calculated by filling in the dots to create a continuous curve.

Head motion correction is another common preprocessing step. When the head moves, the neurons under a voxel move and hence its timecourse now represents largely that of some other voxel in the past. Hence the timecourse curve is effectively cut and pasted from one voxel to another. Motion correction tries different ways of undoing this to see which undoing of the cut-and-paste produces the smoothest timecourse for all voxels. The undoing is by applying a rigid-body transform to the volume, by shifting and rotating the whole volume data to account for motion. The transformed volume is compared statistically to the volume at the first timepoint to see how well they match, using a cost function such as correlation or mutual information. The transformation that gives the minimal cost function is chosen as the model for head motion.

fMRI acquires both many functional images with fMRI and a structural image with MRI. The structural image is usually of a higher resolution and depends on a different signal, the T1 magnetic field decay after excitation. To demarcate regions of interest in the functional image, one needs to align it with the structural one. To figure out which regions the active voxels fall in, one has to align the functional image to the structural one. This is done with a coregistration algorithm that works similar to the motion-correction one, except that here the resolutions are different, and the intensity values cannot be directly compared since the generating signal is different.

To integrate the results across subjects, a common brain atlas can be used, and all the brains are adjusted to align to the atlas, and then analyzed as a single group. The atlases commonly used are the Talairach one and the Montreal Neurological Institute (MNI) one. Alternately, a probabilistic map can be created by combining scans from numerous individuals. This normalization to a standard template is done by mathematically checking which combination of stretching, squeezing, and warping reduces the differences between the target and the reference. While this is conceptually similar to motion correction, the changes required are more complex than just translation and rotation, and hence optimization even more likely to depend on the first transformations in the chain that is checked.

At process block 130, the value of a plurality of striatal connectivity dyads is calculated from the modified scan data obtained in step 120 using an extraction algorithm. A striatal connectivity dyad represents the statistical relationship (correlation) between: 1) the time series of signal data extracted from a region of interest (ROI) within one of 12 subregions within the corpus striatum (centered on the “seed voxels” listed in Table 5); and 2) the time series of signal data extracted from a target ROI within the brain such as those listed in Table 3 and Table 4. Once the regions of interest (ROI) were defined by modifying the scan data, the mean time course of resting state activity was extracted from each seed region, defined as a uniform sphere surrounding the seed voxel. Whole-brain, voxel-wise correlation maps for each ROI, are created, using the extracted waveform as a reference. The resulting correlation maps can then be Fisher's z-transformed. In some embodiments, the seed voxel of one or more of the striatal connectivity dyads is found in a brain region selected from the group consisting of the insula cortex, the opercular cortex, the anterior cingulate, the thalamus, the orbitofrontal cortex, and the precuneus regions. In further embodiments, the plurality of striatal connectivity dyads are selected from the striatal connectivity dyads of Tables 3 and 4.

Process block 140 represents the step of calculating a combined score from the values of the striatal connectivity dyads using a combining algorithm. For example, the inventors extracted the raw connectivity values for the brain regions evaluated for each participant and entered into a large matrix for each cohort. The mean and standard deviation can then be calculated on raw connectivity values, and the first principal component extracted. The raw connectivity values for the connections in the patient datasets can be z transformed by the mean and standard deviation of the corresponding values. The first component's loadings can be used to calculate a factor score for each patient in both the discovery and replication datasets.

The number of striatal connectivity dyads that are calculated and combined can vary in different embodiments of the invention. In some embodiments, the value of at least 20 striatal connectivity dyads are calculated and combined. In other embodiments, the value of at least 40 striatal connectivity dyads are calculated and combined. In further embodiments, the value of at least 60 striatal connectivity dyads are calculated and combined. In yet further embodiments, the value of at least 80 striatal connectivity dyads are calculated and combined.

Process block 150 represents the step of comparing the combined score obtained in step 140 to a classifier value to determine if the subject is a responder or a non-responder. A responder, as used herein, refers to a subject for whom the antipsychotic drug is effective, while a non-responder is a subject for whom the antipsychotic drug is ineffective. An ineffective antipsychotic agent is an agent which has below average activity compared to the typical activity for an antipsychotic agent, while an effective antipsychotic agent is an agent that has average or better activity compared to the typical activity for an antipsychotic agent. The classifier value represents the fMRI image for a subject who has an average response to the antipsychotic agent. A classifier value for the striatal connectivity score can be derived from responder/non-responder status in the discovery cohort, and sensitivity/specificity of this threshold can be tested in the replication cohort in order to determine its clinical utility. If the patient's normalized score is less than the classifier value, then the patient is classified as a responder. If it is greater than or equal to the classifier value, the patient is classified as a non-responder. In some embodiments, the classifier value is 3.8 based on normalized scores derived from 91 striatal connectivity dyads. Accordingly, if the patient's normalized score derived from these 91 dyads is less than 3.8, then he or she is classified as a responder. If greater than or equal to 3.8, the patient is classified by the algorithm as a non-responder. In some embodiments, comparison of the combined score to the classifier value is used to predict length of stay for a subject being administered the antipsychotic agent being evaluated.

In some embodiments, the subject has been diagnosed as having a psychotic disorder. Examples of psychotic disorders include schizophrenia, schizophreniform disorder, schizoaffective disorder, delusional disorder, shared psychotic disorder, brief psychotic disorder, psychotic disorder due to a general medical condition, substance-induced psychotic disorder, bipolar I disorder (with psychotic features) and major depressive disorder (with psychotic features).

In some embodiments, the subject has been diagnosed as having schizophrenia. Schizophrenia is a mental disorder often characterized by abnormal social behavior and failure to recognize what is real. Common symptoms include false beliefs, unclear or confused thinking, auditory hallucinations, reduced social engagement and emotional expression, and lack of motivation. Diagnosis is based on observed behavior and the person's reported experiences. Schizophrenia includes a variety of different subtypes. Examples of subtypes of schizophrenia include paranoid type, disorganized type, catatonic type, undifferentiated type, and residual type.

Antipsychotic agents can be useful for treating conditions other than psychosis. Accordingly, in some embodiments, the method of predicting the response of a subject to an antipsychotic agent is carried out in a subject that has been diagnosed as having a non-psychotic disorder. Examples of non-psychotic disorders that can be treated with psychotic agents include bipolar I disorder (acute treatment of manic, mixed, or depressive episodes; maintenance treatment), major depressive disorder, irritability associated with autistic disorders, agitation associated with schizophrenia or bipolar mania, and irritability associated with autistic disorders.

### Methods for Treatment

In some embodiments, the method of predicting the response of a subject to an antipsychotic agent is used to indicate or guide treatment of a subject. For example, treatment can differ depending on whether or not the subject is identified as a responder or non-responder to the antipsychotic agent. For example, if the subject is identified as a responder, the method can further comprising administering a therapeutically effective amount of a non-clozapine atypical antipsychotic to the subject. Alternately, if the subject is identified as a non-responder, the method can further comprising administering a therapeutically effective amount of clozapine to the subject.

Dosage amounts and schedules for antipsychotic agents are well-known to those skilled in the art. While administration of antipsychotics is the most important aspect in treatment of psychosis, in some embodiments it may be useful to administer other agents, such as those intended to reduce side-effects, while in additional embodiments it may be useful to provide psychosocial interventions such as family therapy, assertive community treatment, supported employment, cognitive remediation, and skills training.

### Systems for Predicting a Response to an Antipsychotic Drug

Another aspect of the invention provides a system 400 for predicting a response of a patient to a given antipsychotic drug. The system includes a medical diagnostic scanner configured to provide a spatial representation of neural activity within the brain; a feature extractor configured to extract a set of striatal connectivity dyads representing the functional connectivity of specified nodes in the basal ganglia to other specified areas of the brain; a classifier configured to classify the patient into one of a plurality of classes representing the likelihood that the patient will respond to the antipsychotic drug from the extracted set of striatal connectivity dyads; and an output device configured to provide the resulting classification to a user in human comprehensible form.

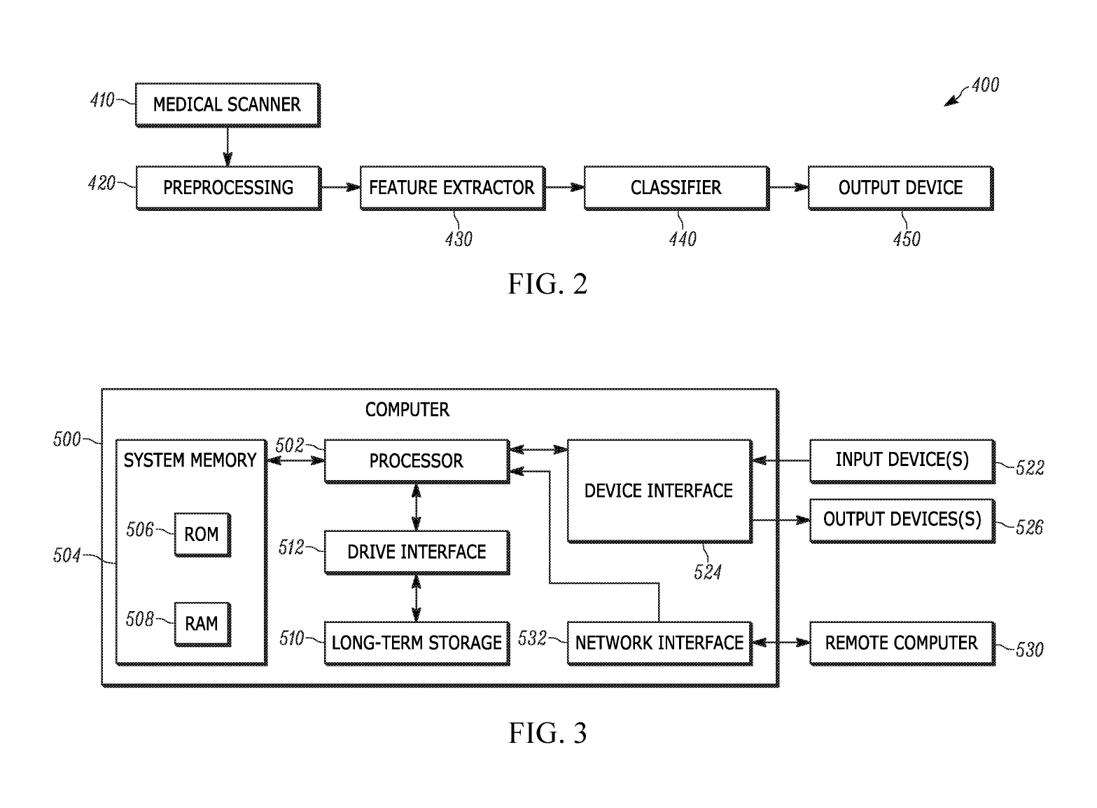

It will be appreciated that the system 400 can be implemented as dedicated hardware, software instructions executed by an associated processor, or a mix of dedicated hardware and software, as shown in FIG. 2. The system 400 includes a medical diagnostic scanner 410 configured to provide a spatial representation of neural activity within the brain. For example, the medical diagnostic scanner 410 can perform a resting-state functional magnetic resonance imaging (rs-fMRI) scan. The scan data is then provided to a preprocessing component 420 that conditions the raw scan data for analysis. The preprocessing component 420, for example, can filter the scan data and standardize it to an appropriate model for feature extraction. In one implementation, the preprocessing component 420 maps the data into a standardized space, such as the Montreal Neurological Institute (MNI) standard brain or the Talairach atlas.

The data is then provided to a feature extractor 430 that extracts a set of striatal connectivity dyads representing the functional connectivity, that is, the statistical correlation of signal intensity patterns, of specified nodes in the basal ganglia to other specified areas of the brain. These connectivity patterns are then provided to a classifier 440 to classify the patient into one of a plurality of classes representing the likelihood that the patient will respond to the drug. In one implementation, the classification is binary, with the patients classified into “responder” or “non-responder” classes. It will be appreciated that the classifier 440 can include one or more of artificial neural networks, support vector machines, rule-based classifiers, statistical classifiers, logistic regression, ensemble methods, decision trees, and other supervised learning algorithms. It will be appreciated that where multiple classification models are utilized in the classifier 440, some form of arbitration, such as a voting scheme, can be provided to provide a final result from the outputs of the multiple models. The resulting classification can be provided to a user in human comprehensible form at an associated output device 450, such as a display.

### Computer Systems

FIG. 3 illustrates a computer system 500 that can be employed to implement systems and methods described herein, such as based on computer executable instructions running on the computer system. The computer system 500 can be implemented on one or more general purpose networked computer systems, embedded computer systems, routers, switches, server devices, client devices, various intermediate devices/nodes and/or stand alone computer systems.

The computer system 500 includes a processor 502 and a system memory 504. Dual microprocessors and other multi-processor architectures can also be utilized as the processor 502. The processor 502 and system memory 504 can be coupled by any of several types of bus structures, including a memory bus or memory controller, a peripheral bus, and a local bus using any of a variety of bus architectures. The system memory 504 includes read only memory (ROM) 506 and random access memory (RAM) 508. A basic input/output system (BIOS) can reside in the ROM 506, generally containing the basic routines that help to transfer information between elements within the computer system 500, such as a reset or power-up.

The computer system 500 can include one or more types of long-term data storage 510, including a hard disk drive, a magnetic disk drive, (e.g., to read from or write to a removable disk), and an optical disk drive, (e.g., for reading a CD-ROM or DVD disk or to read from or write to other optical media). The long-term data storage 510 can be connected to the processor 502 by a drive interface 512. The long-term data storage 510 components provide nonvolatile storage of data, data structures, and computer-executable instructions for the computer system 500. A number of program modules may also be stored in one or more of the drives as well as in the RAM 508, including an operating system, one or more application programs, other program modules, and program data.

A user may enter commands and information into the computer system 500 through one or more input devices 522, such as a keyboard or a pointing device (e.g., a mouse). These and other input devices are often connected to the processor 502 through a device interface 524. For example, the input devices can be connected to the system bus by one or more a parallel port, a serial port or a universal serial bus (USB). One or more output device(s) 526, such as a visual display device or printer, can also be connected to the processor 502 via the device interface 524.

The computer system 500 may operate in a networked environment using logical connections (e.g., a local area network (LAN) or wide area network (WAN) to one or more remote computers 530. A given remote computer 530 may be a workstation, a computer system, a router, a peer device or other common network node, and typically includes many or all of the elements described relative to the computer system 500. The computer system 500 can communicate with the remote computers 530 via a network interface 532, such as a wired or wireless network interface card or modem. In a networked environment, application programs and program data depicted relative to the computer system 500, or portions thereof, may be stored in memory associated with the remote computers 530.

Examples have been included to more clearly describe particular embodiments of the invention. However, there are a wide variety of other embodiments within the scope of the present invention, which should not be limited to the particular example provided herein.

### EXAMPLES

### Example 1

### Striatal Functional Connectivity Predicts Response to Antipsychotic Medications: Findings from Two Independent Cohorts

Our aim was to develop and validate a biomarker that provides a quantitative assay, with clinically useful sensitivity and specificity, predictive of treatment response to widely used first-line antipsychotic medications, based on functional connectivity of the striatum. We created our biomarker in a discovery dataset that consisted of patients with first-episode schizophrenia, and tested our results in a replication dataset comprised of chronic patients with psychotic disorders newly hospitalized for acute psychosis.

### Methods

We trained and tested our biomarker in a step-wise manner. As a proof of concept, we first examined seven specific functional connections that had shown significant longitudinal changes associated with improvement in psychosis from our previous study (Table 3). Sarpal et al., JAMA Psychiatry 72(1):5-13 (2015). We tested whether a single baseline measure of functional connectivity at these key nodes would predict response in our larger discovery cohort of patients undergoing controlled treatment for their first episode of schizophrenia. This was followed by whole-brain mapping of connections from our striatal regions of interest (ROIs) that significantly predicted treatment response in this discovery cohort. Based on the set of results across the whole brain, we computed a prognostic striatal functional connectivity score that was normalized using data from a group of matched healthy participants. We then tested this score in a replication cohort of hospitalized patients who were initiating a trial of antipsychotic medication for treatment of acute psychosis.

### Participants

Our discovery dataset consisted of 41 patients between the ages of 15-40 who were experiencing their first-episode of a schizophrenia spectrum disorder (schizophrenia, schizophreniform disorder, schizoaffective disorder, or psychotic disorder, not otherwise specified), and had two weeks or less of cumulative lifetime exposure to antipsychotic medication. Patients underwent resting state functional MRI (fMRI) scanning and symptom ratings before twelve weeks of treatment with either risperidone or aripiprazole as a part of an NIMH-funded, double blind, randomized control trial (NCT00320671). Assessments with the Clinical Global Impressions Scale (CGI) and the Brief Psychiatric Rating Scale-Anchored (BPRS-A) were performed weekly during the first four weeks, then biweekly for the remaining eight weeks of the study. Treatment response was defined as two consecutive, sustained ratings of a CGI improvement score of much or very much improved, as well as a rating of 3 (“mild”) or less on all of the following items of the BPRS-A: conceptual disorganization, grandiosity, hallucinatory behavior, and unusual thought content.

Our replication dataset consisted of 40 patients hospitalized at The Zucker Hillside Hospital for psychotic symptoms associated with chronic psychotic disorders (CPD). Patients were diagnosed with schizophrenia, schizophreniform disorder, schizoaffective disorder, psychotic disorder, not otherwise specified, or bipolar disorder, type I with psychotic features. Clinical management of these patients followed routine clinical guidelines and was not influenced by our research protocol, but in all cases included antipsychotic medication. Patients underwent resting state fMRI scanning and evaluation of symptoms with the BPRS-A at baseline and weekly during hospitalization. We defined treatment response in this group based on the mean reduction in the combined score of the following psychotic symptoms from the BRPS-A: hallucinations, conceptual disorganization, and unusual thought content. Our mean was calculated to be 30% and this number was used as a threshold for clinical response. Length of stay in the hospital in days was used as a secondary measure of response.

After complete description of the study to all participants, written informed consent was obtained as per a protocol that was approved by the Institutional Review Board of the North Shore-Long Island Jewish Health System.

Resting State fMRI Image Acquisition and Preprocessing

Resting state fMRI scans were collected on a GE 3T scanner. Five minutes of resting-state functional scans (150 whole-brain volumes) were acquired for each study participant. During the scan, participants were asked to close their eyes and instructed not to think of anything in particular. Our preprocessing methods are described in the supplemental materials; notably, strict attention was paid to potential motion artifacts as per the methods described by Power et al. Power et al., NeuroImage 59: 2142-54 (2012).

### Functional Connectivity Analyses

As mentioned above, our aim was to develop a biomarker of treatment response based on functional connectivity between striatal subregions and the rest of brain. To generate this assay, a seed-based functional connectivity approach was applied to the striatum based on the methods of Di Martino et al. Martino et al., Cereb Cortex 18: 2735-47 (2008). Using these methods, we created spherical ROIs, bilaterally, in the dorsal caudate, ventral caudate, nucleus accumbens, dorsal rostral putamen, dorsal caudal putamen, and ventral rostral putamen. Once ROIs were defined, the mean time course of resting state BOLD activity was extracted from each seed region. Whole-brain, voxel-wise correlation maps for each ROI, were then created with the extracted waveform as a reference. The resulting correlation maps were Fisher's z-transformed. Striatal connectivity maps for each striatal ROI showed good correspondence with previous studies that utilized this method.

### Voxel-Wise Survival Analysis

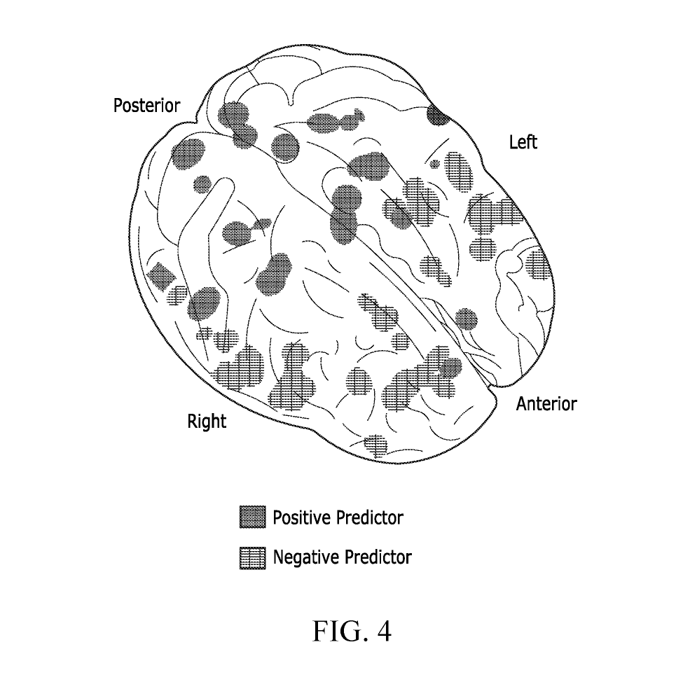

These striatal connectivity z-maps were then utilized to develop our predictive biomarker as follows: (1) for every voxel located within gray matter (181144 voxels total), the corresponding connectivity strength for each first episode patient was entered into a Cox regression analysis along with clinical outcome (response or non-response), and time to outcome (number of weeks); (2) the resulting z scores of this analysis for each voxel were placed in our standard brain space to create whole-brain maps; (3) at the group level we performed one-sample t tests on these maps for each ROI. In order to capture the maximal amount of variance for our predictive biomarker, we applied an exploratory threshold of p<0.005, with cluster size above 9 voxels, to identify striatum-connected nodes potentially relevant to treatment response. At total of ninety-one functional connections across the 12 input ROIs significantly predicted treatment response in either positive or negative directions (FIG. 4, Table 3, Table 4).

### Striatal Functional Connectivity Score Calculation

Next, a whole-brain prognostic assay was computed using the data from our 91 predictive striatal functional connections. In order to reduce circularity in this computation, we normalized data from our discovery group of FES patients with data from healthy comparison (HC) participants matched for age, sex, and education. The raw connectivity values were extracted for all 91 regions for each participant and entered into a large matrix for each of our cohorts. The mean and standard deviation was calculated on raw connectivity values in the HC group, and the first principal component was extracted. The raw connectivity values for each of the 91 connections in our patient datasets were z transformed by the mean and standard deviation of the corresponding values in the HC group. The first component's loadings were used to calculate a factor score for each patient in both the discovery and replication datasets. The resulting score for each participant, which represented the expression of the “healthy” first principal component within the patient's striatal functional connections, was examined for prognostic value against our predetermined response/non-response designation. A cutoff threshold for this striatal connectivity score was derived based on responder/non-responder status in the discovery cohort, and sensitivity/specificity of this threshold were tested in the replication cohort in order to determine clinical utility. The ability of the striatal connectivity score to predict length of stay was also examined in the replication cohort.

### Results

### Demographics

A total of 41 FES patients were included in our discovery cohort. Of this group, 24 patients were classified as responders and 17 were non-responders (Table 1). The healthy control group used for normalization of our prognostic score calculation included 41 participants matched to our FES group for age (mean=21, SD=5.1), sex (29 males, 12 females), and years of education (mean=13.3 years, SD=3.3). Our replication cohort of CPD patients consisted of 40 participants treated with antipsychotics.

TABLE 1
Clinical and demographic information of discovery cohort

| | First-Episode | First-Episode |
|---|---|---|
| | Schizophrenia | Schizophrenia Non- |
| Characteristic | Responders (N = 24) | Responders (N = 17) |
| Age (years) | | |
| Mean | 21.2^{a} | 21.9 |
| SD | 3.8 | 5.9 |
| Gender (number) | | |
| Male | 16^{b} | 13 |
| Female | 8 | 4 |
| Years of Education | | |
| Mean | 12.3^{a} | 12.29 |
| SD | 2.5 | 2.0 |
| Handedness (Edinburgh) | | |
| Mean | 0.70^{a} | 0.79 |
| SD | 0.38 | 0.32 |
| Duration of Untreated | | |
| Psychosis (days) | | |
| Mean | 140^{a} | 110 |
| SD | 269 | 113 |
| Baseline BPRS (total) | | |
| Mean | 44^{a} | 43 |
| SD | 8.3 | 8.2 |

^{a}No significant difference (p < 0.05) from value for comparison group (two-tailed t test).
^{b}No significant difference (p < 0.05) from value for comparison group (Chi-square test).
A priori defined regions

In our previous work with a subsample (n=24) of the first episode cohort with both pre- and post-treatment scans, we discovered seven functional connections of the striatum that increased in connectivity over the course of treatment, in association with decreasing psychotic symptoms. Sarpal et al., JAMA Psychiatry 72(1):5-13 (2015). As a proof of concept, we first tested the prognostic value of these a priori defined regions in our larger first episode cohort (n=41). Baseline connectivity values for these functional nodes were extracted from striatal maps in our FES patients and entered into a series of Cox regression models with time to response as the dependent measure. We found that the strength of the right ventral rostral putamen functional connection to the anterior cingulate significantly predicted response to treatment twelve weeks following resting state scanning (Table 2), even after correcting for multiple comparisons. Two additional connections—right ventral rostral putamen with the thalamus and insula—trended toward significance (Table 2).

TABLE 2
A priori functional connections entered into survival analysis

| Functional Connection | p-value |
|---|---|
| Right dorsal caudate - Dorsolateral prefrontal cortex | 0.7928 |
| Right dorsal caudate - Anterior cingulate | 0.1791 |
| Right dorsal caudate - Orbital frontal cortex | 0.3049 |
| Right dorsal caudate - Thalamus | 0.0814 |
| Right ventral rostral putamen - Anterior cingulate | 0.0027 |
| Right ventral rostral putamen - Insula | 0.0615 |
| Right nucleus accumbens - hippocampus | 0.3803 |

### Whole Brain Predictors

While promising as proof of concept, these associations with a priori regions were not sufficiently powerful to yield a clinically useful biomarker. Consequently, we utilized voxelwise Cox regressions to search for treatment response predictors in striatal connectivity values across the whole brain. We observed 91 connections significantly associated with treatment response (Tables 3 and 4). The insula and opercular cortices, anterior cingulate, thalamus, orbitofrontal cortex, and precuneus were regions that frequently appeared on our list of predictive connections with the striatum. Intriguingly, there was an anterior-posterior gradient in the directionality of the associations (FIG. 4). Posterior regions tended to be positive predictors, meaning greater connectivity of these regions with striatal subdivisions at baseline was associated with better subsequent treatment response. In more frontal regions, we observed prediction in the negative direction; lower striatal connectivity of these nodes at baseline was associated with better subsequent response.

TABLE 3
Significant predictors of response from right hemispheric seeds

| | | | | | Montreal | |
|---|---|---|---|---|---|---|
| | | | | | Neurological | |
| | Direction | | Z | | Institute | |
| Seed | of result | | score | Brodmann | Coordinates | |
| region | (−/+) | k | (max) | Area | (x, y, z) | functional connection |
| VRP | − | 217 | 4.34 | 13 | −38, −2, −2 | Left insular cortex |
| | − | 62 | 3.51 | 11, 25 | 12, 36, −18 | Orbital frontal cortex, subcallosal |
| | | | | | | cortex, medial frontal cortex |
| | − | 62 | 3.42 | | 4, 48, 8 | Anterior Cingulate cortex |
| | − | 25 | 3.45 | 32, 10 | 14, 44, 2 | anterior cingulate cortex |
| | − | 19 | 3.13 | 13 | 38, 0, 0 | Right insular cortex |
| | − | 48 | 3.45 | 11, 25 | −8, 34, −22 | Orbitofrontal cortex, subcallosal |
| | | | | | | cortex, medial frontal cortex |
| | − | 25 | 3.16 | 40 | 56, −20, 26 | Right supramarginal gyrus |
| | − | 15 | 3.07 | 22 | 52, −26, 0 | Superior temporal gyrus |
| | − | 13 | 3.24 | 1 | −66, −14, 20 | Postcentral gyrus |
| | − | 12 | 3.46 | 13 | −26, 14, −20 | Orbital frontal cortex |
| | + | 33 | 3.98 | 9 | −42, 32, 28 | Middle frontal gyrus, dorsolateral |
| | | | | | | prefrontal cortex |
| | + | 10 | 3.08 | 7 | −6, −54, 50 | Precuneus cortex |
| VSI | − | 76 | 3.78 | 39 | −44, −60, 10 | Middle temporal gyrus |
| | − | 89 | 3.77 | 44, 45 | −54, 20, 6 | Inferior frontal gyrus |
| | − | 31 | 3.37 | 9 | −8, 54, 30 | Superior frontal gyrus, |
| | | | | | | paracingulate gyrus |
| | − | 32 | 3.29 | 22 | 50, −24, −2 | Superior temporal gyrus |
| | − | 74 | 3.57 | | −62, −40, −4 | Middle temporal gyrus |
| | − | 9 | 2.95 | 9 | −44, 8, 42 | Middle frontal gyrus |
| | − | 12 | 3.2 | 6 | −8, 4, 64 | Supplemental motor area |
| | − | 9 | 3.02 | | −36, −20, −12 | Hippocampus/parahippocampal |
| | | | | | | gyrus |
| | − | 25 | 3.35 | 9 | 6, 56, 14 | Frontal pole |
| | + | 293 | 4.06 | 7 | 10, −68, 48 | Precuneus cortex |
| | + | 27 | 3.29 | 7 | −8, −68, 50 | Precuneus cortex |
| DC | − | 171 | 4.15 | 6 | 54, 6, 36 | Precentral gyrus |
| | − | 41 | 3.26 | 13, 44 | 54, 8, 2 | Insula, operculum cortex |
| | − | 10 | 3.3 | 45 | −58, 28, 8 | Inferior frontal gyrus |
| | − | 48 | 3.24 | 6 | −54, 8, 14 | Precentral gyrus |
| | − | 36 | 3.26 | | −40, 28, 4 | Frontal opercular cortex |
| | + | 86 | 3.84 | | 8, −40, 18 | Posterior Cingulate? |
| | + | 13 | 3.14 | 19 | −38, −58, 14 | Angular Gyrus |
| DCP | − | 774 | 4.37 | 13, 22, 6 | −42, −4, 6 | Insula, central opercular cortex, |
| | | | | | | precentral gyrus |
| | − | 2.91 | 4.33 | 13, 22 | 48, 12, −2 | Insula, central opercular cortex |
| | − | 24 | 3.31 | NA | −6, −14, 0 | thalamus |
| | − | 11 | 3.16 | 10 | −48, 50, 4 | Frontal pole |
| | − | 16 | 3.01 | | 62, −10, 12 | Supramarginal gyrus |
| | + | 57 | 3.19 | 21 | −60, −46, −4 | Middle temporal gyrus |
| | + | 15 | 3.31 | 9 | −44, 30, 34 | Dorsolateral prefrontal cortex |
| DRP | − | 669 | 4.06 | 13, 22, 6 | −42, −4, 6 | Insula, central opercular cortex, |
| | | | | | | precentral gyrus |
| | − | 211 | 3.63 | 13, 22 | 46, 12, 0 | Insula, central opercular cortex |
| | − | 17 | 3.47 | 8 | 58, 12, 38 | Precentral gyrus |
| | − | 12 | 3.19 | | 28, 20, −16 | Orbitofrontal cortex |
| VSS | − | 165 | 3.61 | | 12, −74, 18 | Posterior cingulate cortex |
| | − | 124 | 4.79 | | −22, −60, 16 | Precuneus cortex |
| | − | 116 | 3.79 | | 52, −38, 14 | Supramarginal gyrus |
| | − | 84 | 3.65 | | −54, −44, 16 | Supramarginal gyrus |
| | − | 59 | 3.5 | | −34, −32, 14 | Planum temporale |
| | − | 53 | 3.32 | | 18, −50, 12 | Precuneus cortex |
| | − | 43 | 3.27 | | 44, 50, 2 | Frontal pole |
| | − | 29 | 2.94 | | 54, −36, 34 | Supramarginal gyrus |
| | − | 20 | 3.39 | | 16, −26, 12 | Thalamus |
| | − | 16 | 3.23 | | −14, −24, 8 | Thalamus |
| | − | 11 | 3.17 | | 10, 22, 30 | Anterior cingulate |

TABLE 4
Significant predictors of response from left hemispheric seeds

| | | | | | Montreal | |
|---|---|---|---|---|---|---|
| | | | | | Neurological | |
| | Direction | | Z | | Institute | |
| Seed | of result | | score | Brodmann | Coordinates | |
| region | (−/+) | k | (max) | Area | (x, y, z) | Functional connection |
| VRP | − | 15 | 3.21 | 25, 11 | 12, 36, −18 | Orbital frontal cortex |
| | − | 15 | 3.04 | 13 | −40, −2, −2 | Insula |
| | − | 10 | 2.97 | 25 | 6, 20, −18 | Subcollosal cortex |
| | + | 74 | 4.25 | NA | 24, −26, 8 | Thalamus |
| | + | 29 | 3.07 | NA | −16, −26, 10 | Thalamus |
| | + | 11 | 3.41 | 46 | −42, 32, 26 | Dorsolateral prefrontal cortex |
| VSI | − | 29 | 3.78 | 39 | −44, −58, 10 | Middle temporal gyrus |
| | − | 24 | 3.26 | 44 | −54, 20, 4 | Inferior frontal gyrus |
| | + | 56 | 3.48 | 7 | −14, −68, 46 | Superior parietal lobule |
| | + | 52 | 3.44 | NA | −12, −14, 8 | Thalamus |
| | + | 12 | 2.89 | | 54, −38, 48 | Supramarginal gyrus |
| | + | 10 | 3.08 | | 16, −74, 44 | Lateral occipital |
| | | | | | | cortex/Precuneus |
| DC | − | 18 | 3.13 | 37 | −54, −64, −8 | Inferior temporal gyrus |
| | − | 22 | 3.2 | 11 | 24, 36, −16 | Frontal pole, orbital frontal cotex |
| | − | 10 | 3.2 | NA | −22, 16, 2 | Putamen |
| | − | 32 | 3.49 | NA | 6, 18, 2 | Accumbens, caudate |
| | + | 100 | 3.98 | 41 | −36, −32, 14 | Planum temporale |
| | + | 25 | 3 | 18 | 10, −76, 18 | occipital cortex |
| | + | 21 | 3.26 | 41 | 46, −36, 14 | Planum temporale |
| | + | 20 | 3.25 | 6 | −16, 22, 58 | Superior frontal gyrus |
| DCP | − | 63 | 3.41 | 4, 43, 13 | −58, −2,14 | Insula, opercular cortex, |
| | | | | | | precentral gyrus |
| | − | 114 | 3.62 | 4, 43, 13 | 48, 8, 0 | Insula, opercular cortex, |
| | | | | | | precentral gyrus |
| | − | 119 | 3.3 | 13 | −42, −8, 4 | Insula |
| | − | 16 | 3.21 | 43 | 60, −6, 12 | planum polare |
| | + | 43 | 3.7 | 9 | −40, 30, 28 | Dorsolateral prefrontal cortex |
| DRP | − | 49 | 3.65 | 13 | 50, 12, 0 | Frontal operculum cortex, |
| | | | | | | interior frontal gyrus |
| | − | 54 | 3.09 | 13 | −42, −8, 4 | Insula, heschl's gyrus |
| | − | 11 | 3.78 | 11 | 16, 34, −14 | Orbital frontal cortex, medial |
| | | | | | | frontal cortex |
| | − | 15 | 3.22 | 32, 9 | 12, 58, 12 | Anterior cingulate cortex, |
| | | | | | | paracingulate gyrus |
| | + | 22 | 3.39 | 21 | −62, −48, 2 | Middle temporal gyrus |
| | + | 12 | 3.13 | 9 | −42, 30, 28 | Middle frontal gyrus |
| VSS | − | 60 | 3.3 | NA | −18, 18, −10 | Accumbens, putamen, caudate |
| | − | 13 | 3.13 | NA | 4, 16, 0 | Acumbens, caudate |
| | − | 12 | 3.32 | 6 | −34, 4, 34 | Middle frontal gyrus |
| | + | 70 | 3.77 | NA | 16, −26, 12 | Thalamus |
| | + | 50 | 3.2 | | −36, −22, 20 | Insula |
| | + | 45 | 3.27 | NA | −10, −18, 12 | Thalamus |
| | + | 40 | 3.32 | 18 | 16, −76, 18 | Occipital cortex |
| | + | 18 | 3.41 | | 44, −38, 14 | Supramarginal gyrus |

### Prognostic Training

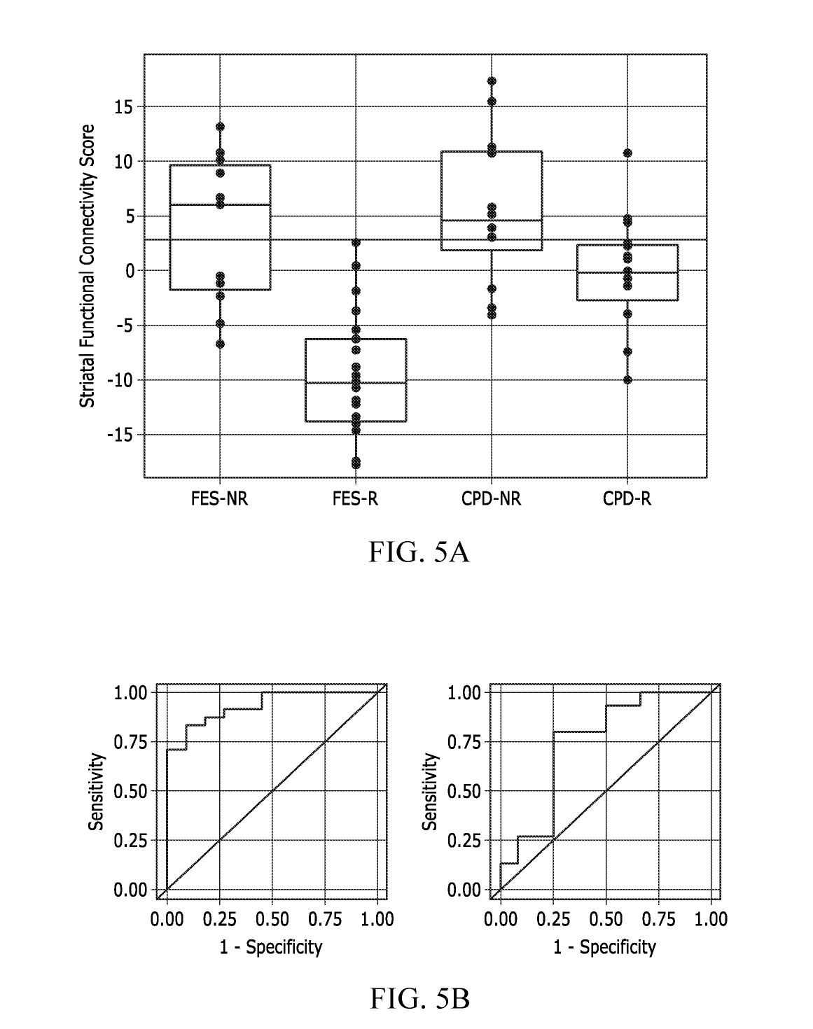

To derive our prognostic index, combining information from all 91 connections into a single score, we normalized the raw connectivity values using our group of HC participants. Loadings from the first principal component of the functional connectivity values of our 91 connections in the HC were calculated in our FES group. As shown in FIG. 5, a score based on these loading was plotted against our predetermined response/non-response status, excluding six subjects who dropped out of the trial within the first two weeks (i.e., before an adequate trial had been attained). Not surprisingly, our test separated responders and non-responders (p=4×10⁻⁵), with lower scores associated with subsequent response. A cutoff score placed just above the highest-scoring responder provided the optimal cutoff, but of course it should be noted that sensitivity and specificity of this cutoff are confounded by the fact that this index was initially derived from analysis of this same cohort.

### Replication

In order to independently replicate the association between striatal connectivity score and outcome, and to determine the sensitivity and specificity of our cutoff threshold in a real-world clinical setting, we applied these methods to our replication cohort of chronic psychosis patients under treatment with antipsychotic medications. Our assay showed a significant separation between responders and non-responders (p=0.026). In the associated receive operating curve, we observed an 80% sensitivity and 75% specificity for prediction (FIG. 5C).

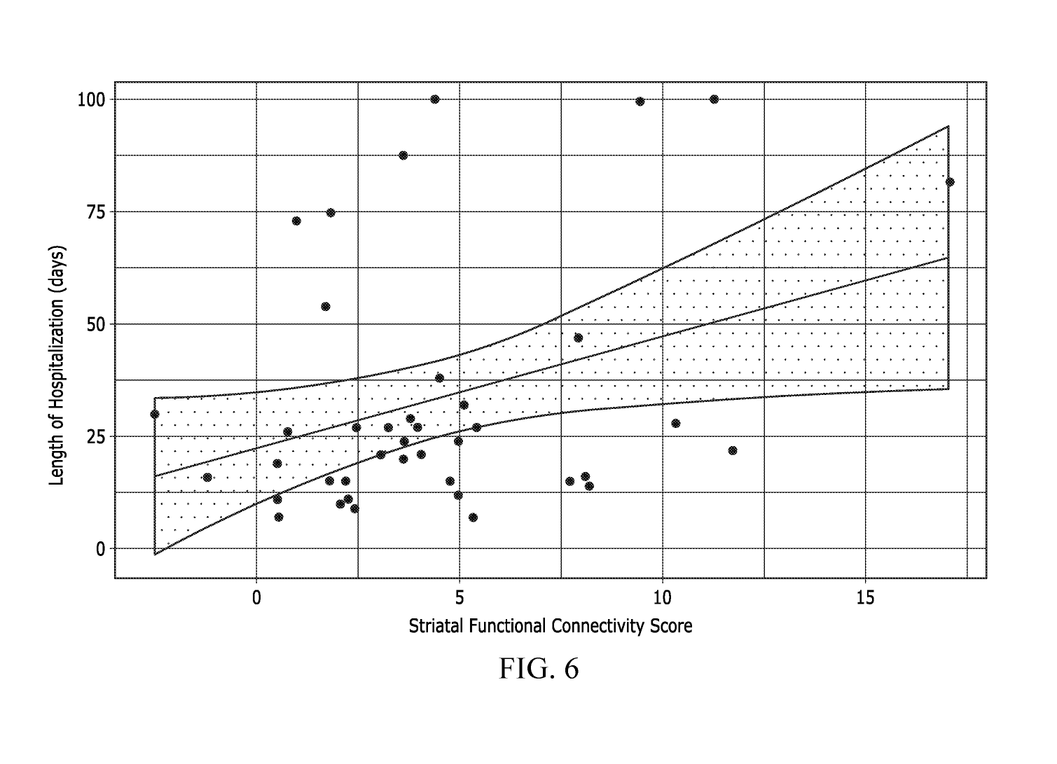

As a secondary analysis, we plotted our score against length of stay in the hospital, as shown in FIG. 6. The median length of stay in the hospital was found to be 24 days, ranging from 7 to 235 days. There was a significant association between length of stay in the hospital and our score (R²=0.11, p=0.02). To illustrate that extreme outliers in the length of the stay analysis do not bias our results, we re-calculated all statistics after winsorization of the data; values higher than 100 days were substituted with 100. Our winsorized results remained significant (R²=0.11, p=0.029).

### Discussion

In the present study, we devised a pre-treatment, fMRI-based biomarker that predicts response to treatment with antipsychotic medications in patients with psychotic disorders. As a proof of concept we were able to extend our previous work by showing that functional connections of the striatum, which demonstrate treatment-related changes, can also provide prognostic information. We subsequently identified striatal functional connectivity nodes significantly associated with treatment response in a discovery dataset of patients with FES, and developed a prognostic score normalized using a group of matched HC participants. We then applied this measure to a replication cohort of CPD patients undergoing treatment for psychotic symptoms. In both our discovery and replication datasets, we observed a significant separation between responders and non-responders with clinically meaningful levels of sensitivity and specificity. In addition, our biomarker correlated with length of stay for psychotic symptoms in a large psychiatric treatment facility.

While there has been an abundance of research in psychotic disorders utilizing neuroimaging techniques recently, including numerous studies of resting state functional connectivity, there remains a crucial gap between these studies and clinical practice. Resting state fMRI has provided insight into the intrinsic functional make-up of psychotic disorders, but has yet to offer clinical utility. This method has been examined as a prognostic measure in depression, as well as chronic pain, but has not yet been reported to predict treatment outcome in psychotic disorders. Chen et al., Biol Psychiatry 62: 407-14 (2007); Baliki et al. Nat Neurosci 15: 1117-9 (2012). Clinical assays derived from the method we describe have the potential to tease apart the clinical heterogeneity of psychotic disorders, and guide treatment real-world treatment decisions.

To our knowledge, our study is the first to report an fMRI-based neuroimaging method with direct clinical applications that can be integrated with existing therapeutic approaches. For example, clozapine is a treatment reserved for patients who are refractory to standard antipsychotic agents. Prior to initiation of this drug, patients have often endured years of untreated illness, with severe psychotic symptomatology, and have experienced a significant impairment in their functioning. Additionally, non-adherence to care has been shown to be associated with increased rates of relapse and worsened outcomes for patients, and lack of initial efficacy is a prominent cause of non-adherence. Moreover, patients who do not remit exhibit increases in violent behavior, and need for emergent care. Prognostic indicators such as the one we report here have the potential to ease the burden incurred by refractory illness. This could have a potentially major impact for patients, families and clinical care providers.

The burden of refractory illness has effects on health care systems as well. The economic cost associated with refractory illness is substantial. Reports indicate that total health care utilization cost estimates for treatment refractory schizophrenia are 3 to 11 fold higher than in patients who respond to standard treatments. Kennedy et al., Int Clin Psychopharmacol 29: 63-76 (2014). Treatment-resistant patients may account for 60-80% of the total cost associated with schizophrenia. Identifying these patients sooner and adjusting our approaches to care with available resources may reduce the economic ramifications of these disorders. Our finding relating our prognostic score to length of stay in a hospital setting also has the potential to predict load on health care systems and ultimately reduce costs.

Biomarkers such as the one we describe may also assist in the development of novel treatments for patients. Earlier recognition of non-response to treatment and addressing the heterogeneity associated with antipsychotic treatment may enhance the quality of clinical trials involving novel agents. Subdividing our population of patients based upon an assay rooted in the underlying biology of illness also opens a door for personalized approaches to psychiatric care. It also has the potential to reduce the ambiguity that associated with medication choices in current practice and lead to more efficient medication trials.

Our results also provide further insight into the mechanisms that underlie psychotic symptoms. It has been theorized that the pathophysiology of psychosis is associated with abnormal assignment of salience to external stimuli. Coordination between salience and executive networks is thought to mediate salience processing, possibly by matching internal states and presumptions with external stimuli. The component regions of the salience network, including the insula and anterior cingulate, have been shown to be dysfunctional in schizophrenia. Palaniyappan et al., Neuron 79: 814-28 (2013). Many of the functional connections of the striatum that predict response to treatment in our analysis are with regions within the salience network. Overall we observed lower striatal functional connectivity scores in responders, and higher scores in non-responders. Our results indicate that deficits in connectivity between the striatum and the salience network may be a target of antipsychotic treatment, consistent with our prior study of treatment mechanisms. By contrast, non-responders tended to have greater striatal connectivity with these salience-network regions in frontal cortex, suggesting an alternative mechanism for their psychosis that is impervious to the primary functional effects of standard antipsychotic medications.

In addition, our results may relate to findings that show differences in dopaminergic tone within the striatum. Further studies are required to investigate if there is a correlation between higher functional connectivity between the striatum and areas within the salience network and normo-dopaminergic capacity in the striatum. Both of these findings correlate with treatment non-response, and may represent two related findings specific to a subgroup of patients with psychosis. Conversely, lower a lower striatal functional connectivity score in our analysis may be associated with hyper-dopaminergia.

Future work is desirable to further characterize our prognostic measurement. Limitations of our analysis include access to a relatively limited number of patients in the two cohorts. Combinations between our test and other biologically based biomarkers such as pharmacogenomics measures and genetic loading for illness may enhance our results. Finally, it will be useful to determine how our assay works in the context of treatment with clozapine, which has markedly different clinical properties compared to all other antipsychotics.

To summarize, we describe a biomarker for response to antipsychotic medication in patients entering treatment of psychosis. This assay may be the first of its kind to provide clinical utility. Furthermore incorporation of such a measure into the clinical practice of prescribers has the potential to decrease the overall suffering of patients, families, and strain on our health care systems.

### Example II

### Additional Methods and Results for Striatal Functional Connectivity Analysis

### Methods

### Participants

All patients received physical examination and laboratory screening to rule out medical causes for their psychotic symptoms. Patients in this group received double blind treatment with either risperidone (dose range: 1-6 mg) or aripiprazole (5-30 mg) for twelve weeks. Simultaneous treatment with mood stabilizers or antidepressants was not allowed, thought patients were treated with diphenhydramine or benztropine as needed for extrapyramidal symptoms, and lorazepam for akathisia, agitation, and anxiety. Patient diagnoses were based on the Structured Clinical Interview for Axis I Diagnostic and Statistical Manual-IV Disorders (SCID). Clinical raters were blind to medication status and trained according to our standardized NIMH protocol (P50MH080173).

Exclusion criteria for all study participants included magnetic resonance imaging contraindications, neurologic conditions (Gilles de la Tourette's, Huntington's Disease, Parkinson's Disease, encephalitis, strokes, aneurysms, tumors, central nervous system infections or degenerative brain diseases), and any serious medical disorder that could affect brain functioning or the participant's capacity to provide informed consent.

Exclusion criteria for the HC group included present use of any psychotropic medications, and the presence of any lifetime history of a major mood or psychotic disorder as determined by clinical interview using the SCID, Non-Patient edition.

### Resting State Scanning and Preprocessing

We used a GE Signa HDx scanner. In each scan session, an anatomical scan was acquired in the coronal plane using an inversion-recovery prepared 3D fast spoiled gradient (IR-FSPGR) sequence (TR=7.5 ms, TE=3 ms, TI=650 ms matrix=256×256, FOV=240 mm) that produced 216 contiguous images (slice thickness=1 mm), comprising a total of 150 echo-planner imaging (EPI) volumes with the following parameters: TR=2000 ms, TE=30 ms, matrix=64*64, FOV=240 mm, slice thickness=3 mm, 40 continuous axial oblique slices (one voxel=3.75×3.75×3 mm). All participants were spoken to between scan sequences to ensure they were not asleep, and no behavioral differences were observed between groups during scanning.

For preprocessing of resting-state scans, FMRIB Software Library (FSL) at the Oxford Centre for Functional MRI of the Brain and Analysis of Functional Neurolmages (AFNI) at the National Institute of Mental Health based scripts were used. The first four EPI volumes were discarded. Each participant's structural image was normalized by a 12-parameter affine transformation to MNI152 space. This transformation was then applied to each individual's functional dataset. Rigid body motion correction was performed with FLIRT and skull stripping was performed with BET. Images were spatial smoothed with a 6-mm FWHM Gaussian kernel. The resulting time series was then high-, and low-pass filtered at 0.05 Hz and 0.1 Hz, respectively. For removal of nuisance variables, each individual's 4D time series data were regressed with eight predictors in a general linear model: white matter (WM), cerebrospinal fluid (CSF), and six motion parameters. To avoid interference with our connectivity measures, the global mean was not included in this calculation.

### Motion Correction

Both relative and absolute motion displacement were examined for each resting state scan. Head motion was calculated as a scalar quantity by the empirical formula detailed in Power et al. (Power et al., Neurolmage 59: 2142-54 (2012), and similarly for the other rigid body parameters. Rotational displacement was calculated by displacement on the surface of a sphere of radius 50 mm, which is approximately the mean distance from the cerebral cortex to the center of the head. The distribution of frame-wise displacement was compared between groups by using an independent Welch t-test. Additionally we performed a group-wise comparison of the derivative of the root mean squared variance (DVARS), which indexes the rate of change of BOLD signal across the entire brain at each frame of data. We used Thomas Nichols' script to calculate standardized DVARS.

### Functional Connectivity

Our ROIs were 3.5 mm spherical regions around a seed voxel (Table 5). AFNI (3dfim+) was used to create our functional maps.

TABLE 5
Seed Voxel Coordinates

| Seed | MNI coordinates |
|---|---|
| Dorsal caudate | x = ±13, y = 15, z = 9 |
| Ventral caudate | x = ±10, y = 15, z = 0 |
| Nucleus accumbens | x = ±9, y = 9, z = −8 |
| Dorsal rostral putamen | x = ±25, y = 8, z = 6 |
| Dorsal caudal putamen | x = ±28, y = 1, z = 3 |
| Ventral rostral putamen | x = ±20, y = 12, z = −3 |

### Score Calculation

We calculated our prognostic score in the R statistical environment using the procedure of the R Project for Statistical Computing.

### Results

### Demographics

Eighteen patients with FES were treated with aripiprazole and 22 patients were treated with risperidone. No significant differences were found in the distribution of these medications between FES responders and non-responders.

Our CPD group consisted of patients with the following diagnoses: BP with psychotic mania (n=11), schizophrenia (n=10), schizophreniform disorder (n=3), psychotic disorder not otherwise specified (n=5), schizoaffective disorder (n=11). The mean age for this group was 29.0 (SD=11.4), 29 were males, 11 were females, and the mean number of year of education was 13.3 (SD =1.9). These patients underwent treatment with the following antipsychotic medications: aripiprazole, asenapine, clozapine, fluphenazine, haloperidol, lurasidone, olanzapine, paliperidone, perphenazine, quetiapine, risperidone.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of predicting the response of a subject to an antipsychotic agent, comprising the steps of:
a) Obtaining functional MRI (fMRI) scan data of the brain of the subject;
b) Modifying the scan data using a standardizing algorithm to provide modified scan data;
c) Calculating the value of a plurality of striatal connectivity dyads from the modified scan data using an extraction algorithm;
d) Calculating a combined score from the values of the striatal connectivity dyads using a combining algorithm; and
e) Comparing the combined score to a classifier value to determine if the subject is predicted to be a responder or a non-responder.

2. The method of claim 1, further comprising the step of obtaining the fMRI scan data by conducting an fMRI scan of the subject using an fMRI imaging apparatus.

3. The method of claim 1, wherein the subject has been diagnosed as having a psychotic disorder selected from the group consisting of schizophrenia, schizophreniform disorder, schizoaffective disorder, delusional disorder, shared psychotic disorder, brief psychotic disorder, psychotic disorder due to a general medical condition, substance-induced psychotic disorder, bipolar I disorder (with psychotic features) and major depressive disorder (with psychotic features).

4. The method of claim 1, wherein the subject has been diagnosed as having schizophrenia.

5. The method of claim 1, wherein the subject has been diagnosed as having a non-psychotic disorder selected from the group consisting of bipolar I disorder (acute treatment of manic, mixed, or depressive episodes; maintenance treatment), major depressive disorder, Irritability associated with autistic disorders, agitation associated with schizophrenia or bipolar mania, and irritability associated with autistic disorders.

6. The method of claim 1, wherein the value of at least 20 striatal connectivity dyads are calculated and combined.

7. The method of claim 1, wherein the value of at least 40 striatal connectivity dyads are calculated and combined.

8. The method of claim 1, wherein the value of at least 60 striatal connectivity dyads are calculated and combined.

9. The method of claim 1, wherein the value of at least 80 striatal connectivity dyads are calculated and combined.

10. The method of claim 1, wherein the plurality of striatal connectivity dyads are selected from the striatal connectivity dyads of Tables 3 and 4.

11. The method of claim 1, wherein the seed voxel of one or more of the striatal connectivity dyads is found in a brain region selected from the group consisting of the insula cortex, the opercular cortex, the anterior cingulate, the thalamus, the orbitofrontal cortex, and the precuneus regions.

12. The method of claim 1, further comprising administering a therapeutically effective amount of a non-clozapine atypical antipsychotic to the subject if the subject is predicted as a responder.

13. The method of claim 1, further comprising administering a therapeutically effective amount of clozapine to the subject if the subject is predicted as a non-responder.

14. A system for predicting a response of a patient to a given antipsychotic drug comprising:
a medical diagnostic scanner configured to provide a spatial representation of neural activity within the brain;
a feature extractor configured to extract a set of striatal connectivity dyads representing the functional connectivity of specified nodes in the basal ganglia to other specified areas of the brain;
a classifier configured to classify the patient into one of a plurality of classes representing the likelihood that the patient will respond to the antipsychotic drug from the extracted set of striatal connectivity dyads; and
an output device configured to provide the resulting classification to a user in human comprehensible form.

15. The system of claim 14, further comprising a preprocessing component configured to condition the spatial representation of neural activity within the brain for analysis.

16. The system of claim 15, wherein the preprocessing component maps the data into the Montreal Neurological Institute standard brain.

17. The system of claim 14, wherein the medical diagnostic scanner is configured to perform a functional magnetic resonance imaging scan.

18. The system of claim 14, wherein the classifier performs a binary classification, such that the patients classified into a “responder” or “non-responder” class.
